(19) United States
(12) Patent Application Publication
(10) Pub. No.: US 2024/0355481 A1
(43) Pub. Date: Oct. 24, 2024

(54) SYSTEM AND METHOD FOR WEATHER-BASED EVALUATION OF EXPOSURE TO AIRBORNE TOXINS TO NEARBY RESIDENTS

(71) Applicants: Amy Rosmarin, North Salem, NY (US); David R. Brown, Westport, CT (US)

(72) Inventors: Amy Rosmarin, North Salem, NY (US); David R. Brown, Westport, CT (US)

(21) Appl. No.: 18/634,898

(22) Filed: Apr. 13, 2024

Related U.S. Application Data
(60) Provisional application No. 63/497,542, filed on Apr. 21, 2023.

(51) Int. Cl. G16H 50/30; G01W 1/00

(52) U.S. Cl. CPC G16H 50/30; G01W 1/00

(57) ABSTRACT

Described are systems and methods configured to accurately identify risk levels for individuals with varying sensitivities for those in close proximity to an emitting source and illustrates the impact of weather on levels of toxic concentration of airborne chemicals. As the methods and implementations described herein can be used for any location and any polluting point source of airborne chemicals, it can be a valuable tool to help residents in their efforts to reduce exposures and for medical providers to more accurately diagnose and treat exposed patients. For various distances from a polluting source, this methodology integrates weather, air dispersion analysis and emissions data of a single chemical or mixture of chemicals to provide a quantitative estimate of the frequency of unhealthy exposures to airborne toxins. Moreover, it specifies the frequency and past hours of unhealthy exposures according to the exposed person's sensitivity level.

### BACKGROUND OF THE DISCLOSURE

Industrial sources emit airborne pollutants that impact health. Concentrations of these pollutants near emitting facilities vary according to local weather conditions but can frequently be high—especially at night. People in close proximity to toxin-emitting facilities are frequently exposed to health-hazardous air.

There is a need to protect the tens of millions of residents in the US who live, work or study near facilities with toxic emissions. According to the FracTracker Alliance this includes an estimated 17.3 million people within ½ mile of oil and gas facilities, of which 5.7 million are people of color [1].

Many more live ½ mile or further from other facilities that also emit large quantities of toxins into the air and are frequently exposed to concentrations of toxins at levels hazardous to their health.

### SUMMARY OF THE DISCLOSURE

The following briefly describes embodiments to provide a basic understanding of some aspects of the innovations described herein. This brief description is not intended as an extensive overview. It is not intended to identify key or critical elements, or to delineate or otherwise narrow the scope. Its purpose is merely to present some concepts in a simplified form as a prelude to the more detailed description that is presented later.

Described herein is a method and systems and apparatuses therefor configured to accurately identify risk levels for individuals with varying sensitivities for those in close proximity to an emitting source and illustrates the impact of weather on levels of toxic concentration. As the methods and implementations described herein can be used for any location and any polluting point source, it can be a valuable tool to help residents in their efforts to reduce exposures.

At least one embodiment is a method implemented by a computer including a processor, and a memory including program memory including instructions for executing the methods described above and herein.

At least one embodiment is a computer program product including program memory including instructions which, when executed by processor, executes the methods described above and herein.

### BRIEF DESCRIPTION OF THE DRAWINGS

Non-limiting and non-exhaustive embodiments are described with reference to the various figures unless otherwise specified.

For a better understanding, reference will be made to the following Detailed Description, which is to be read in association with the accompanying drawings, wherein:

FIG. 1 shows levels of an individual's sensitivity to natural gas VOC emissions.

FIGS. 2A-2B are tables showing a frequency of exposure to an unhealthy concentration of VOCs in 2020.

FIGS. 3A-3E are tables showing the frequency of exposure to an unhealthy concentration of VOCs by wind direction in 2020.

FIG. 4 is a chart showing cumulative frequency of consecutive exposure hours.

FIG. 5 is a chart showing the observed peak frequency from a monitor and frequency of high exposure hours based on the weather model.

FIG. 6 is a chart showing a number of daily hourly peaks by weather model and monitor for a period of months.

FIG. 7 is a chart showing 24 hour average monitor readings compared to single hour readings.

FIG. 8 is a system diagram of an environment in which at least one of the various embodiments can be implemented.

FIG. 9 is a cloud computing architecture in accordance with at least one of the various embodiments.

FIG. 10 is a logical architecture for a cloud computing platform in accordance with at least one of the various embodiments.

FIG. 11 is a system flow for a display interface that can render a display of the information produced by the components of the system.

FIGS. 12A-12E show formulas for performing the calculations for Exposure Frequency Risk

### DETAILED DESCRIPTION OF THE EMBODIMENTS

Various embodiments now will be described more fully hereinafter with reference to the accompanying drawings, which form a part hereof, and which show, by way of illustration, specific embodiments by which the innovations described herein can be practiced. The embodiments can, however, be embodied in many different forms and should not be construed as limited to the embodiments set forth herein. Rather, these embodiments are provided so that this disclosure will be thorough and complete, and will fully convey the scope of the embodiments to those skilled in the art. Among other things, the various embodiments can be methods, systems, media, or devices. The following detailed description is, therefore, not to be taken in a limiting sense.

Throughout the specification and claims, the following terms take the meanings explicitly associated herein, unless the context clearly dictates otherwise. The term “herein” refers to the specification, claims, and drawings associated with the current application. The phrase “in one embodiment” or “in an embodiment” as used herein does not necessarily refer to the same embodiment or a single embodiment, though it can. Furthermore, the phrase “in another embodiment” as used herein does not necessarily refer to a different embodiment, although it can. Thus, as described below, various embodiments can be readily combined, without departing from the scope or spirit of the present disclosure.

In addition, as used herein, the term “or” is an inclusive “or” operator, and is equivalent to the term “and/or” unless the context clearly dictates otherwise. The term “based on” is not exclusive and allows for being based on additional factors not described, unless the context clearly dictates otherwise. In addition, throughout the specification, the meaning of “a” “an” and “the” include plural references. The meaning of “in” includes “in” and “on.”

As used herein, the term “Host” can refer to an individual person, partnership, organization, or corporate entity that can own or operate one or more digital media properties (e.g., web sites, mobile applications, or the like). Hosts can arrange digital media properties to use hyper-local targeting by arranging the property to integrate with widget controllers or servers.

### Illustrative Operating Environment

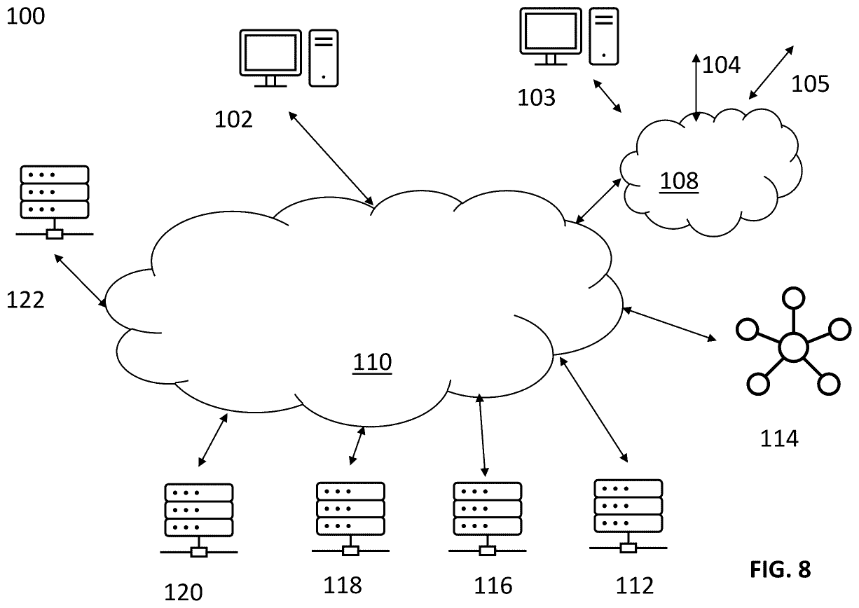

FIG. 8 shows components of one embodiment of an environment in which embodiments of the innovations described herein may be practiced. Not all of the components may be required to practice the innovations, and variations in the arrangement and type of the components may be made without departing from the spirit or scope of the innovations. As shown, system 100 of FIG. 8 includes local area networks (LANs)/wide area networks (WANs)—(network) 110, wireless network 108, client computers 102-105, Service Provider Platform Server Computer 112, Financial Service Entity Server 116, Content Data Server 118, Registry Server 120, Exchange Server 122, and Distributed Immutable Ledger Server Computers 114.

In one embodiment, at least some of client computers 102-105 may operate over a wired and/or wireless network, such as networks 110 and/or 108. Generally, client computers 102-105 may include virtually any computer capable of communicating over a network to send and receive information, perform various online activities, offline actions, or the like. In one embodiment, one or more of client computers 102-105 may be configured to operate within a business or other entity to perform a variety of services for the business or other entity. For example, client computers 102-105 may be configured to operate as a web server, an analytics server, an ingress or egress server, or the like. However, client computers 102-105 are not constrained to these services and may also be employed, for example, as an end-user computing node, in other embodiments. It should be recognized that more or less client computers may be included within a system such as described herein, and embodiments are therefore not constrained by the number or type of client computers employed.

Computers that may operate as client computer 102-105 may include computers that typically connect using a wired or wireless communications medium such as personal computers, multiprocessor systems, microprocessor-based or programmable electronic devices, network PCs 102, 103, or the like. In some embodiments, client computers 102-105 may include virtually any portable personal computer capable of connecting to another computing device and receiving information such as, laptop computer 104 and smart mobile telephone 105, and tablet computers, and the like. However, portable computers are not so limited and may also include other portable devices such as cellular telephones, display pagers, radio frequency (RF) devices, infrared (IR) devices, Personal Digital Assistants (PDAs), handheld computers, wearable computers, integrated devices combining one or more of the preceding devices, and the like. As such, client computers 102-105 typically range widely in terms of capabilities and features. Moreover, client computers 102-105 may access various computing applications, including a browser, or other web-based application.

A web-enabled client computer may include a browser application that is configured to receive and to send web pages, web-based messages, and the like. The browser application may be configured to receive and display graphics, text, multimedia, and the like, employing virtually any web-based language, including a wireless application protocol messages (WAP), and the like. In one embodiment, the browser application is enabled to employ Handheld Device Markup Language (HDML), Wireless Markup Language (WML), WMLScript, JavaScript, Standard Generalized Markup Language (SGML), HyperText Markup Language (HTML), extensible Markup Language (XML), and the like, to display and send a message. In one embodiment, a user of the client computer may employ the browser application to perform various activities over a network (online). However, another application may also be used to perform various online activities.

Client computers 102-105 may also include at least one other client application that is configured to receive and/or send content between another computer. The client application may include a capability to send and/or receive content, or the like. The client application may further provide information that identifies itself, including a type, capability, name, and the like. In one embodiment, client computers 102-105 may uniquely identify themselves through any of a variety of mechanisms, including an Internet Protocol (IP) address, a phone number, Mobile Identification Number (MIN), an electronic serial number (ESN), or other device identifier. Such information may be provided in a network packet, or the like, sent between other client computers, Platform Server Computer 112, Distributed Immutable Ledger Database Server Computer 114, or other computers.

Client computers 102-105 may further be configured to include a client application that enables an end-user to log into an end-user account that may be managed by another computer, such a Server Computer 112, Distributed Immutable Ledger Computers 114, or the like. Such end-user account, in one non-limiting example, may be configured to enable the end-user to manage one or more online activities, including in one non-limiting example, search activities, social networking activities, browse various websites, communicate with other users, or the like. However, participation in such online activities may also be performed without logging into the end-user account.

Wireless network 108 is configured to couple client computers 103-105 and its components with network 110. Wireless network 108 may include any of a variety of wireless sub-networks that may further overlay stand-alone ad-hoc networks, and the like, to provide an infrastructure-oriented connection for client computers 103-105. Such sub-networks may include mesh networks, Wireless LAN (WLAN) networks, cellular networks, and the like. In one embodiment, the system may include more than one wireless network.

Wireless network 108 may further include an autonomous system of terminals, gateways, routers, and the like connected by wireless radio links, and the like. These connectors may be configured to move freely and randomly and organize themselves arbitrarily, such that the topology of wireless network 108 may change rapidly.

Wireless network 108 may further employ a plurality of access technologies including 2nd (2G), 3rd (3G), 4th (4G) 5th (5G) generation radio access for cellular systems, WLAN, Wireless Router (WR) mesh, and the like. Access technologies such as 2G, 3G, 4G, 5G, and future access networks may enable wide area coverage for mobile devices, such as client computers 103-105 with various degrees of mobility. In one non-limiting example, wireless network 108 may enable a radio connection through a radio network access such as Global System for Mobil communication (GSM), General Packet Radio Services (GPRS), Enhanced Data GSM Environment (EDGE), code division multiple access (CDMA), time division multiple access (TDMA), Wideband Code Division Multiple Access (WCDMA), High Speed Downlink Packet Access (HSDPA), Long Term Evolution (LTE), and the like. In essence, wireless network 108 may include virtually any wireless communication mechanism by which information may travel between client computers 103-105 and another computer, network, and the like.

Network 110 is configured to couple network computers with other computers and/or computing devices, including, Platform Server Computers 112, Distributed Immutable Ledger Server Computers 114, client computer 102, and client computers 103-105 through wireless network 108. Network 110 is enabled to employ any form of computer readable media for communicating information from one electronic device to another. Also, network 110 can include the Internet in addition to local area networks (LANs), wide area networks (WANs), direct connections, such as through a universal serial bus (USB) port, other forms of computer-readable media, or any combination thereof. On an interconnected set of LANs, including those based on differing architectures and protocols, a router acts as a link between LANs, enabling messages to be sent from one to another. In addition, communication links within LANs typically include twisted wire pair or coaxial cable, while communication links between networks may utilize analog telephone lines, full or fractional dedicated digital lines including T1, T2, T3, and T4, and/or other carrier mechanisms including, for example, E-carriers, Integrated Services Digital Networks (ISDNs), Digital Subscriber Lines (DSLs), wireless links including satellite links, or other communications links known to those skilled in the art. Moreover, communication links may further employ any of a variety of digital signaling technologies, including without limit, for example, DS-0, DS-1, DS-2, DS-3, DS-4, OC-3, OC-12, OC-48, or the like. Furthermore, remote computers and other related electronic devices could be remotely connected to either LANs or WANs via a modem and temporary telephone link. In one embodiment, network 110 may be configured to transport information of an Internet Protocol (IP). In essence, network 110 includes any communication method by which information may travel between computing devices.

Additionally, communication media typically embodies computer readable instructions, data structures, program modules, or other transport mechanism and includes any information delivery media. By way of example, communication media includes wired media such as twisted pair, coaxial cable, fiber optics, wave guides, and other wired media and wireless media such as acoustic, RF, infrared, and other wireless media.

Platform Server Computer 112 includes virtually any network computer capable of supporting Applications and Application Program Interfaces therefor as well as providing network and scoring tools as describe herein. Computers that may be arranged to operate as Platform Server Computer 112 include various network computers, including, but not limited to personal computers, desktop computers, multiprocessor systems, microprocessor-based or programmable consumer electronics, network PCs, server computers, network appliances, and the like.

Although FIG. 8 illustrates Platform Server Computer 112 as a single computer, the invention is not so limited. For example, one or more functions of the Platform Server Computer 112 may be distributed across one or more distinct network computers. Moreover, Platform Server Computer 112 is not limited to a particular configuration. Thus, in one embodiment, Platform Server Computer 112 may contain a plurality of network computers. In another embodiment, Platform Server Computer 112 may contain a plurality of network computers that operate using a master/slave approach, where one of the plurality of network computers of Platform Server Computer 112 is operative to manage and/or otherwise coordinate operations of the other network computers. In other embodiments, the Platform Server Computer 112 may operate as a plurality of network computers arranged in a cluster architecture, a peer-to-peer architecture, and/or even within a cloud architecture. Other configurations, and architectures are also envisaged.

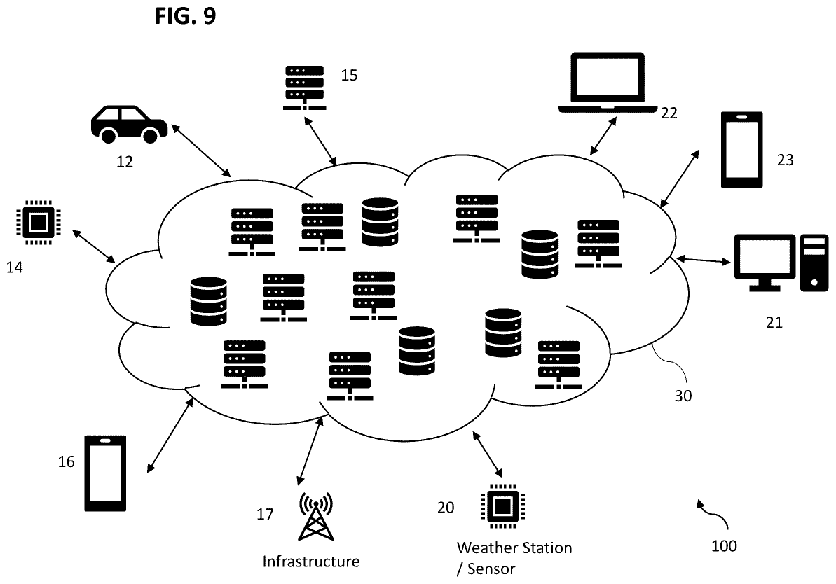

FIG. 9 is a logical architecture of system 100 for weather exposure risk processing and analytics in accordance with at least one embodiment.

As described herein, embodiments of the system 100, processes and algorithms can be configured to run on a web services platform host such as Amazon Web Services (AWS)® or Microsoft Azure®. A cloud computing architecture is configured for convenient, on-demand network access to a shared pool of configurable computing resources (e.g. networks, network bandwidth, servers, processing, memory, storage, applications, virtual machines, and services). A cloud computer platform can be configured to allow a platform provider to unilaterally provision computing capabilities, such as server time and network storage, as needed automatically without requiring human interaction with the service's provider. Further, cloud computing is available over a network and accessed through standard mechanisms that promote use by heterogeneous thin or thick client platforms (e.g., mobile phones, laptops, and PDAs). In a cloud computing architecture, a platform's computing resources can be pooled to serve multiple consumers, partners or other third party users using a multi-tenant model, with different physical and virtual resources dynamically assigned and reassigned according to demand. A cloud computing architecture is also configured such that platform resources can be rapidly and elastically provisioned, in some cases automatically, to quickly scale out and rapidly released to quickly scale in.

Cloud computing systems can be configured with systems automatically control and optimize resource use by leveraging a metering capability at some level of abstraction appropriate to the type of service (e.g., storage, processing, bandwidth, and active user accounts). Resource usage can be monitored, controlled, and reported.

A cloud computing architecture includes a number of service and platform configurations.

A Software as a Service (SaaS) is configured to allow a platform provider to use the provider's applications running on a cloud infrastructure. The applications are accessible from various client devices through a thin client interface such as a web browser (e.g., web-based e-mail). The consumer typically does not manage or control the underlying cloud infrastructure including network, servers, operating systems, storage, or even individual application capabilities, with the possible exception of limited user-specific application configuration settings.

A Platform as a Service (PaaS) is configured to allow a platform provider to deploy onto the cloud infrastructure consumer-created or acquired applications created using programming languages and tools supported by the provider. The consumer does not manage or control the underlying cloud infrastructure including networks, servers, operating systems, or storage, but can a have control over the deployed applications and possibly application hosting environment configurations.

An Infrastructure as a Service (IaaS) is configured to allow a platform provider to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

A cloud computing architecture can be provided as a private cloud computing architecture, a community cloud computing architecture, or a public cloud computing architecture. A cloud computing architecture can also be configured as a hybrid cloud computing architecture comprising two or more clouds platforms (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure comprising a network of interconnected nodes.

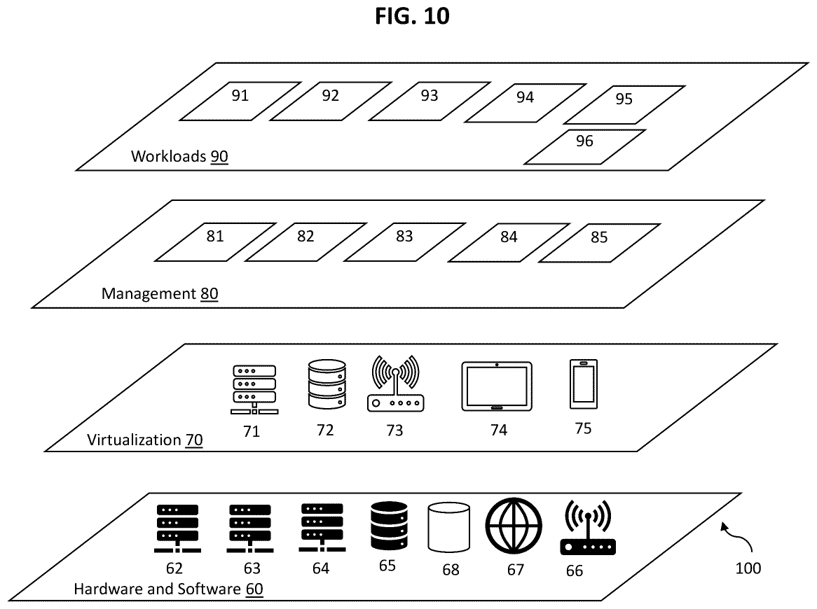

As shown in FIG. 9, cloud computing environment 50 comprises one or more cloud computing nodes 30 with which local computing devices used by cloud consumers, such as, for example, personal digital assistant (PDA) or cellular telephone 23, desktop computer 21, laptop computer 22, and sensor event data source 14, application data source 16, weather data source 20, wireless infrastructure data source 17, and third party data source 15 and/or automobile computer systems such as vehicle data source 12. Nodes 30 can communicate with one another. They can be grouped (not shown) physically or virtually, in one or more networks, such as private, community, public, or hybrid clouds as described herein, or a combination thereof. The cloud computing environment 50 is configured to offer infrastructure, platforms and/or software as services for which a cloud consumer does not need to maintain resources on a local computing device. It is understood that the types of computing devices shown in FIG. 10 are intended to be illustrative only and that computing nodes 30 and cloud computing environment 50 can communicate with any type of computerized device over any type of network and/or network addressable connection (e.g., using a web browser).

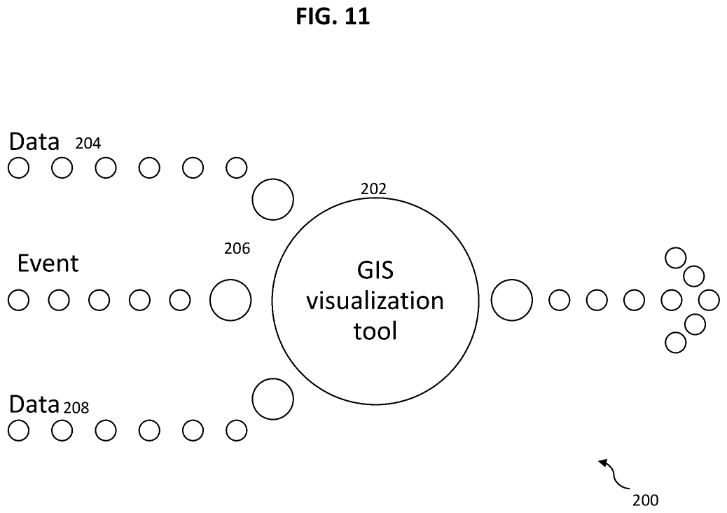

Referring now to FIG. 10, a set of functional abstraction layers provided by cloud computing environment 50 (FIG. 9) is shown. The components, layers, and functions shown in FIG. 11 are illustrative, and embodiments as described herein are not limited thereto. As depicted, the following layers and corresponding functions are provided:

A hardware and software layer 60 can comprise hardware and software components. Examples of hardware components include, for example: mainframes 61; servers 62; servers 63; blade servers 64; storage devices 65; and networks and networking components 66. In some embodiments, software components include network application server software 67 and database software 68.

Virtualization layer 70 provides an abstraction layer from which the following examples of virtual entities can be provided: virtual servers 71; virtual storage 72; virtual networks 73, including virtual private networks; virtual applications and operating systems 74; and virtual clients 75.

In one example, management layer 80 can provide the functions described below. Resource provisioning 81 provides dynamic procurement of computing resources and other resources that are utilized to perform tasks within the cloud computing environment. Metering and Pricing 82 provide cost tracking as resources are utilized within the cloud computing environment, and billing or invoicing for consumption of these resources. In one example, these resources can comprise application software licenses. Security provides identity verification for cloud consumers and tasks, as well as protection for data and other resources. User portal 83 provides access to the cloud computing environment for consumers and system administrators. Service level management 84 provides cloud computing resource allocation and management so that required service levels are met. Service Level Agreement (SLA) planning and fulfillment 85 provides pre-arrangement for, and procurement of, cloud computing resources for which a future requirement is anticipated in accordance with an SLA.

Workloads layer 90 provides examples of functionality for which the cloud computing environment can be utilized. Examples of workloads and functions that can be provided from this layer include mapping 91; sensor data processing 92, event data processing 93; portal dashboard delivery 94; data analytics processing 95; and other workload processing 96.

Although this disclosure describes embodiments on a cloud computing platform, implementation of embodiments as described herein are not limited to a cloud computing environment.

One of ordinary skill in the art will appreciate that the architecture of system 100 is a non-limiting example that is illustrative of at least a portion of an embodiment. As such, more or less components can be employed and/or arranged differently without departing from the scope of the innovations described herein. However, system 100 is sufficient for disclosing at least the innovations claimed herein.

The present disclosure implements technology based on a number of key findings from work undertaken in conjunction with the implementations described herein:

People in close proximity to toxin-emitting facilities are frequently exposed to unhealthy air.

The EPA's regional National Ambient Air Quality Standards (NAAQS) do not reveal the high levels of toxins for people in close proximity to emitting facilities.

A weather-based model as described herein estimate exposure levels for those in close proximity to polluting facilities.

In an implementation, historical hourly weather data was input and Pasquill air dispersion calculations performed to quantitatively model the dispersion of natural gas compressor station airborne VOCs. The objective was to estimate hourly air concentration levels and therefore health risk at discreet distances near an emitting source. A continuous VOC air monitor was used to confirm the weather model's results. Based on EPA methodology and NIOSH data, in an implementation, the data was processed to produce VOC Exposure Frequency Risk charts showing risk levels for individuals with varying sensitivities.

A system configured to employ this disclosures' analytical methodology can be implemented at any emission site, as is demonstrated in this disclosure using one year of data at a single compressor station location.

For people living close to an emitting facility, the projected toxic concentration in the air from facility emissions frequently exceed acceptable chemical risk. These risks however, are not revealed by the EPA's regional NAAQS. NAAQS masks the toxicity to those in close proximity because its procedures average toxic concentration over longer times and regional areas and silos chemicals. Accordingly, described is a system, apparatus, and method therefor that can advantageously be used to accurately project toxic concentration and chemical Exposure Frequency Risk for individuals with varying sensitivities.

### Limitations of the EPA's NAAQS Pollution Assessments

The EPA's NAAQS are the basis for the federal government and most states' emissions regulations [3-5]. For example, while the EPA has established an environmental risk ranking for VOCs, it does not have a health standard for VOCs. Therefore, NAAQS focus is on average regional emissions of 8 or 24 hours. Because NAAQS average the air pollution regionally and over time it does not reflect the toxicity of the air for people living in close proximity to emitting facilities. NAAQS' measurements under-estimate the acute risks and thereby fail to identify the need to protect nearby residents and the need to reduce the amount of VOCs emitted. Consequently, NAAQS and emission regulations do not adequately protect the health of millions of people.

### Acute and Chronic Health Effects of VOCs

Although outdoor air quality has improved in many parts of the USA in the 50 years since the passing of the Clean Air Act, current pollutant levels still present major localized health risks. In 2020, it was estimated that outdoor air pollution caused from between 100,000 and 200,000 US premature deaths a year [6]. The following pollutants are estimated to be associated with total US premature pollution-related deaths: PM 2.5 41%, NO₂ 19%, NH3 17%, VOCs 12%, and sulphur oxides 10% [6]. Economic costs from air pollution-related illness are estimated at $150 to $790 billion per year [7-10].

Experts have identified a statistically significant positive association between exposure to air pollution such as PM2.5, NO₂, and VOCs and incidence, severity and mortality of Covid-19 resulting both from health impacts due to long term exposure as well as from level of current exposure [11-12]. One large study in 33 European nations linked higher outdoor VOCs with significantly higher rates of Covid-19 incidence and mortality [13].

VOC exposures are associated with health effects such as many cancers/neoplasms, blood and immune system, endocrine and related, mental and behavioral, nervous system, eye and adnexa, ear and mastoid process, circulatory, respiratory including asthma, digestive, skin and subcutaneous, genitourinary: urinary, pelvis, genitals and breasts, congenital malformations and chromosomal abnormalities, symptoms, signs, abnormal clinical and laboratory findings. [14-20].

Those who are within a few miles of the emitting facility are most at risk. While there are limited health studies concerning health risk to high peak exposures lasting an hour or less, the Harvard Six Cities study and the American Cancer Society study [21-22] of particulate air pollution and mortality indicate that such exposures occur frequently and that people who live near sites emitting VOCs and other toxins and who are highly sensitive to short term exposures, such as those with asthma and COPD, are at risk.

Statistical studies show exposures to low to moderate concentrations of VOCs are associated with adverse health effects including respiratory symptoms, neuropsychiatric symptoms and increased risk of cancer [23]. A number of studies have noted that low level concentrations of VOCs can worsen asthma and other breathing problems. A meta-analysis of 49 published studies links low level indoor VOC levels to increased risk of asthma and wheezing [23].

Exposure to higher concentrations have been shown to produce immediate respiratory, neurological and cardiovascular effects. A study of fifth grade children in Kanawha County, West Virginia reported that increased levels of outdoor volatile chemicals as low as 2 μg/m3 were associated with significant increases in chronic respiratory symptoms [17]. A case-control study in Perth, Australia reported that indoor exposure to many VOCs such as benzene, toluene, xylene and total VOCs were significantly higher in 88 young children (6 months to 3 years) with asthma as compared to 104 controls [24]. A representative study of 550 US adults reported that low level exposure of many VOCs including benzene, ethylbenzene, 2,4 dichlorobenzene, and MTBE (methyl tetra butyl ether) were associated with significantly higher asthma rates [25].

A review of VOC studies reported that 1 μg/m3 of many VOCs including benzene, toluene, xylene, acetaldehyde, and p-dichlorobenzene are associated with significantly increased risk of specific adverse health effects including asthma, leukemia, cardiovascular diseases, and adverse birth outcomes including low birth weight. Results of some of these meta-analysis are listed below [14]. A study of indoor VOCs in Louisiana reported that levels of VOCs can affect pulmonary function in asthmatics and may cause unacceptable lifetime cancer risks. Exposures may be unacceptable for benzene, carbon tetrachloride, and chloroform [16].

Health Concerns and Exposures Associated with Natural Gas Facilities

Natural gas production, transportation and use are major sources of airborne pollutants. VOCs and other pollutants are emitted into the air in large quantities annually from the power plants, compressor stations, processing plants, well pads, and leaking pipelines, as well as from industrial and manufacturing facilities that span the nation in urban, suburban and rural locations [26]. Conventional and non-conventional gas production are major producers of VOCs and other air pollutants including CO2, PM 2.5, NO₂, and at least 39 known human carcinogens including arsenic, lead and numerous polycyclic aromatic hydrocarbons (PAHs) [26-30]. Studies have reported significant levels of various VOCs located near natural gas facilities including compressor stations [18-20].

The chemicals in the emissions from natural gas infrastructure are linked to 19 of 20 major categories of disease states including pulmonary, cardiovascular, endocrine and neurological conditions, birth defects and cancer [26]. One study of three homes located from 0.8 to 1.7 km from a natural gas compressor station reported that levels of many VOCs including benzene, toluene, xylenes, ethyl benzene and 1,2,4 trichlorobenzene were significantly elevated above the 1 μg/m3, a threshold of unhealthy levels [19].

Significantly higher levels of many pollutants in the air, water, and soil near natural gas producing regions are reported [29, 31-33]. Major air pollutants produced by fracking include PM_{2.5}, methane, VOCs, NO₂, as well as other hazardous air pollutants including benzene, toluene, ethyl benzene, xylenes, formaldehyde, hydrogen sulfide, and polycyclic aromatic hydrocarbons [24].

Mckenzie reported that levels of VOCs were significantly increased near fracking well sites—especially within ½ mile. Exposure to some VOCs such as 1-3 butadiene and benzene exceeded 1 per million lifetime cancer risk [34]. Air emissions of polycyclic aromatic PAHs are often considerable [35].

The health effects of mixtures of air pollutants including VOCs, NO₂, PM2.5 have not been as extensively studied as studies analyzing health effects of individual chemicals [36]. The synergistic health impacts of multiple toxins, however, is established [37]. Peng et al recently described a method to estimate health effects of mixtures of wildfire air pollutants by using methods such as inverse regression, propensity score matching, and principal stratification [36]. It is probable that the health risks of mixtures of chemicals are greater than suggested by the majority of studies which only analyze one pollutant.

Helmig reported that VOCs and NO₂ emissions from fracking may play a significant role in ground level ozone formation in Colorado [38].

Studies have linked indoor or outdoor air exposure in areas near fracking operations to a number of adverse health effects [31-32]. A 2019 review of 20 epidemiological studies reported significant positive associations with adverse health conditions and fracking in 15 studies [31]. This review reported positive associations between fracking and adverse birth outcomes, leukemia, and tumors CNS, bladder and thyroid, cardiovascular hospitalization, psychological problems and asthma [31].

Brown reported that in southwest Pennsylvania proximity to unconventional natural gas developments reported proximity-related respiratory symptoms, cough, and shortness of breath [39]. Bushong also reported a significant association between unconventional gas production and increased asthma hospitalizations in Pennsylvania counties [40].

A southwest Pennsylvania study indicated that some symptoms (eyes, ears, nose, throat; neurological and muscular) may be associated with proximity to fracking operations [41]. Unconventional natural gas production has been associated with a wide range of negative health effects including adverse birth outcomes [42-45], childhood blood cancers [46], sinusitis/headache/fatigue [47], increased traffic accidents [48], depression and disordered sleep [49], and increased hospitalization rates [50].

A study that estimated the health risk of exposure to fracking operations for residents living near oil and gas fracking facilities in Colorado measured 56 VOCS and concluded non-cancer risks for all individual compounds were well below standards, however the hazard indexes were slightly above 1 [51]. Lifetime exposure cancer risks were between 1.5 to 3.6×10⁻⁵ for benzene and 7.3×10⁻⁶ for ethyl benzene [51].

### Methodology

An objective of this disclosure's analysis was to identify the frequency of disproportionately high exposures of over one hour to airborne toxins emitted by a nearby polluting facility and to quantify the impact on persons of different sensitivities to acute health effects. The disclosure's weather-based methodology estimated the number of hours, days and nights that residents at various distances from a toxic emitter would potentially be breathing contaminated air hazardous to their health.

When pollutants are released into outdoor air, in addition to the amount of the pollutants emitted and the toxicity of the individual and/or mix of chemicals, five factors determine the concentration of the toxins in nearby air and the resulting inhalation exposures at downwind locations:
1. The amount of time the air mass is over the source
2. The amount of dilution that occurs downwind at various wind speeds
3. The degree of dilution due to vertical mixing which is determined by degree of cloud cover and subsequent surface temperature differentials
4. Wind direction
5. Distance from the source of the emission

The actual health impact from each exposure to an individual is determined by the sensitivity of the exposed person. Greater concentration and more frequent exposures have greater risk of impacts.

The disclosure employed calculations where the number of hours, days and nights that local weather conditions were such that VOC mixtures from an emitting facility would contaminate the air 0.1 km to 10 km from that facility. The findings were compared to the health effects from the mixture of VOCs emitted.

Hourly 2020 weather data from the National Oceanic and Atmospheric Association (NOAA) was inputted. That data was then overlaid onto air dispersion charts based on Pasquill air dispersion graphs [52-53] that calculate toxin concentration at discreet distances. The charts developed in the disclosure specify the frequency at the discreet distances that the toxin concentration exceeds acceptable risk for people of varying health conditions and therefore varying sensitivities to toxins. Pasquill's airborne chemical dispersion graphs were developed in World War 2 so troops could assess the toxic concentration of chemical warfare [53-54]. Currently they are the seminal logic extensively used by the nuclear, coal/gas power stations and many other industries to evaluate emissions' safety [53-54]. The EPA uses Pasquill's information as the basis for their air quality measurements.

An analysis was performed on historical weather data for Danbury Airport [55], which is 3½ miles E-SE from the mid-sized, Title V fracked gas compressor station used in this disclosure. Annual emissions of 40,000 lbs of VOCs—the amount that this compressor station emitted in 2020 [56] was plotted. Annual emissions of 20,000 lbs to evaluate the extent of exposure to toxins from facilities with less emissions was also plotted.

In a related analysis, the hours that were identified as meeting the criteria for being unhealthy were compared to the hours that showed peak levels of VOCs as determined by a continuous air monitor. An hour-by-hour analysis of the disclosure's weather model's alert-hours and no alert hours indicated that they were the same hours as the monitor's peak and no peak hours 87% of time.

### Exposure Guidance for VOC Risk

The objective of EPA regulatory limits on VOCs is to establish VOC air levels that limit concentrations of ground level ozone for regulatory actions rather than to evaluate direct VOC impact on human health. There are, however, well-referenced exposure standards and some indoor air risk standards for specific VOCs. This information was used to develop the risk ranking for exposures to the actual mixture of VOC emissions evaluated in this disclosure. Emissions from compressor stations include mixtures of the same VOCs.

### Weather Model Basis

In order to determine frequency of acute risk, the number of times the concentration of VOCs in the air would exceed the health recommendations for people of varying sensitivities based on EPA methodology and NIOSH data for the specific mixture of VOCs present was calculated. The analysis utilized publicly available hourly 2020 weather data. The analysis indicated the number of times in a year during the day and separately during the night that the weather conditions were such that a person living near a natural gas compressor station, power plant or other natural gas toxin-emitting facility at that location annually emitting 40,000 lbs and 20,000 lbs of VOCs could be breathing air that is hazardous to their health.

Utilizing the local historical weather data, the analysis selected as its basis the annual VOC emissions from a medium-sized gas transmission pipeline compressor station. As reported to the New York State Department of Environmental Conservation (NYS DEC), this facility emitted 40,000 lbs of VOCs in 2020.

This disclosure assumed identical hour emissions rate for the year. Actual emissions fluctuate with some hours resulting in a higher concentration of toxins in the air and some hours a lower concentration. The methodology, however, can reflect differing emissions if, as one example, emissions are greater in winter months.

### Developing Exposure Frequency Charts

Utilizing information from Pasquill graphs [52-53], this disclosure's analysis categorized in its charts the actual frequency of high VOC concentrations. Risks levels are expressed by color-code in the charts to reflect risk by resident sensitivity level. The risks are consistent with air quality index criteria for VOC pollution and integrate wind direction, wind speed, cloud cover, day-part and distance from the emitting facility to estimate the toxin concentration in the air. The analysis categorized the hourly NOAA data as follows:

| | |
|---|---|
| Wind direction | North, South, East, West |
| Wind speed | <5 mph, 5-7 mph, 8-11 mph, >11 mph |
| Cloud cover | Clear, <50%, >50% |
| Day-part | Day 6am-6pm, Night 6pm-6am |
| Date/time | Actual date/time |

The methodology can use wind directions other than North, South, East and West. It could use windspeed categories other than <5 mph, 5-7 mph, 8-11 mph, >11 mph. It could use cloud cover categories other than Clear, <50%, >50%. It could use different hours to define Day and Night.

It was noted that when there is no wind, (0 wind direction), the air is somewhat stagnant. Consequently, for those hours, the data were applied to all four directions. At night it was applied to those less than 1 km (0.6 miles) away from the emitting source. During the day when there was at least some cloud cover it similarly was applied in all 4 directions to those less than 1 km away from the source. While 1 km was used in the disclosure, this methodology can include a distance that is not equal to 1 km. During the day when it was clear and sunny, the toxins were assumed to dilute vertically and so those hours were not included in the analysis.

For hours when wind direction data was identified by NOAA as variable, the wind direction and speed in the hours before and after were examined visually and the variable hour's wind direction was manually attributed to the direction that appeared to reflect the predominant wind direction. Allocation of variable wind direction can be done by computer model

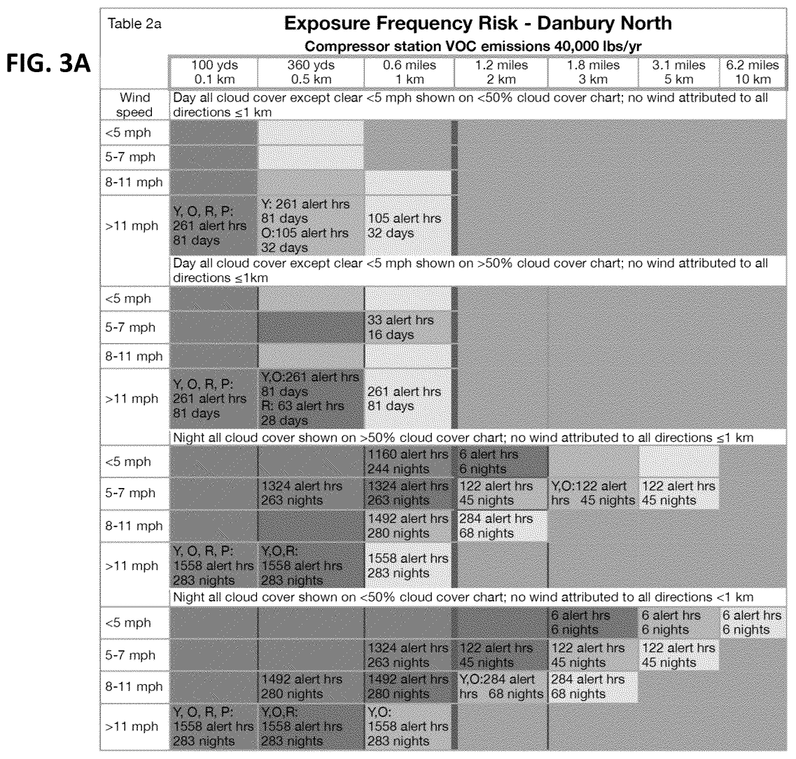

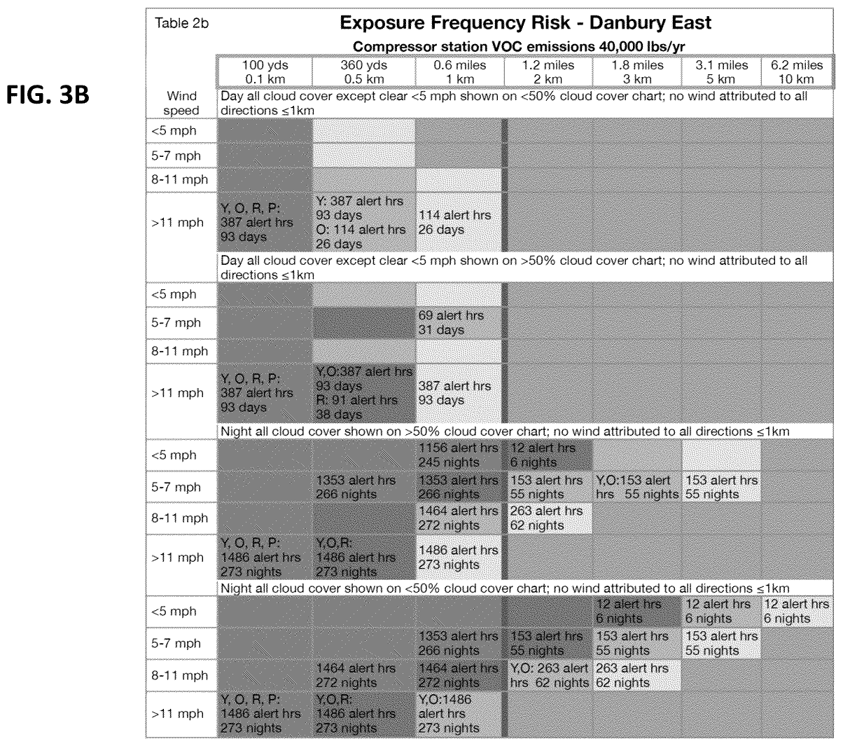

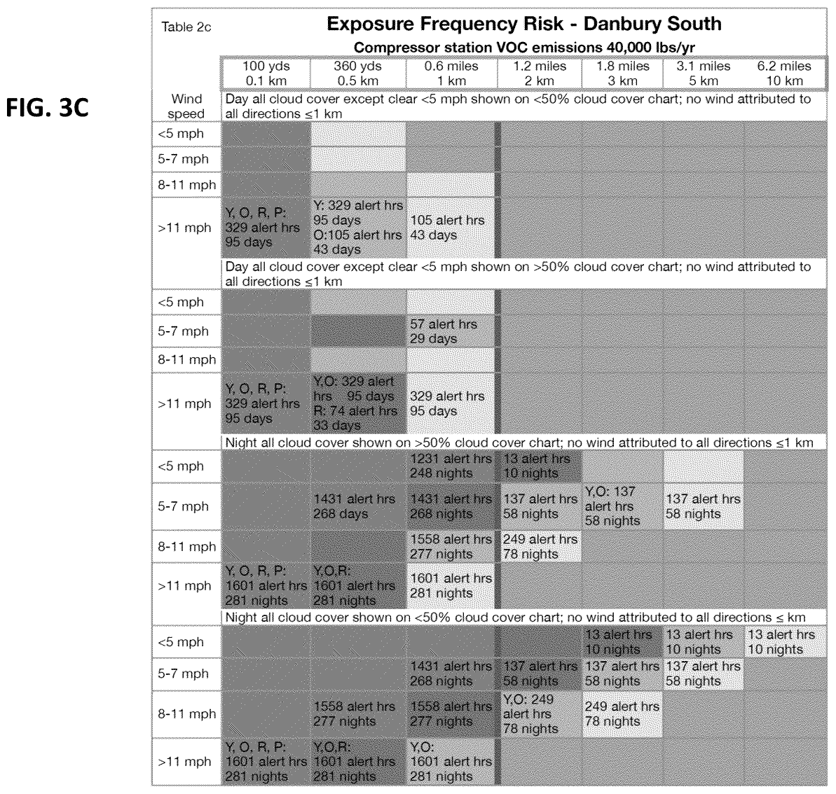

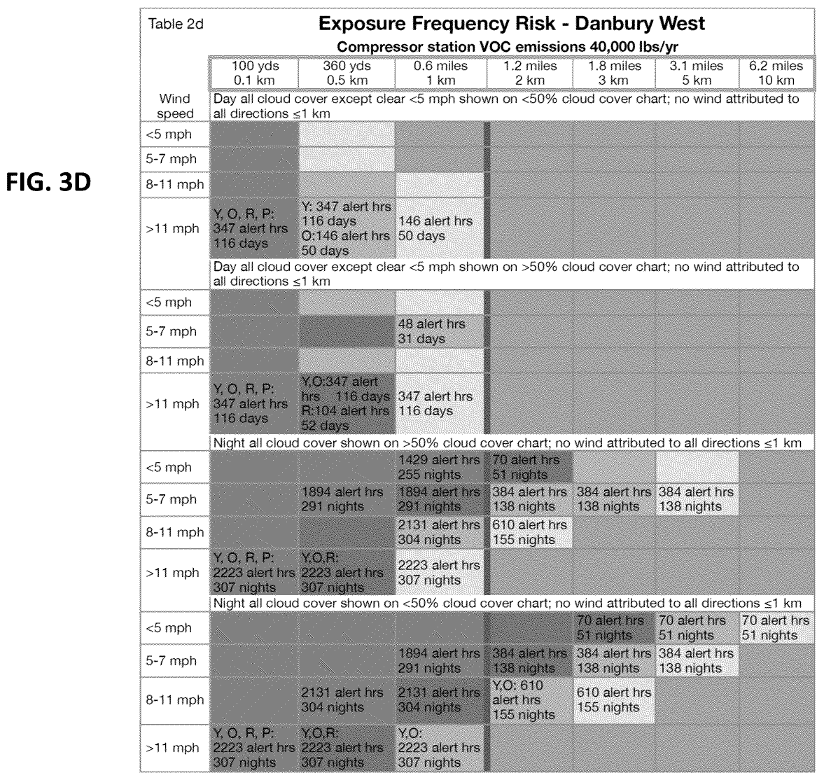

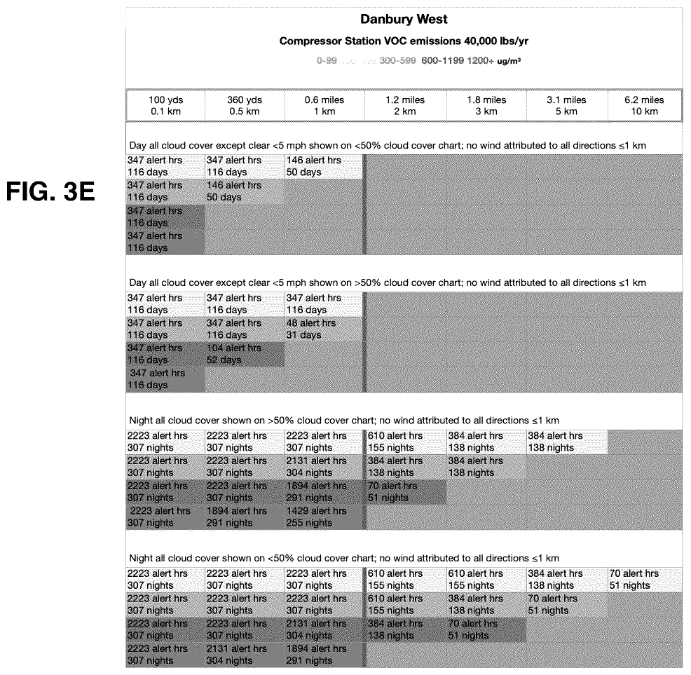

As shown in FIGS. 3A-3E, for each wind direction and day-part, two charts show the disclosure's Exposure Frequency Risk. One chart reflects dispersion with less than 50% cloud cover and one reflects dispersion with greater than 50% cloud cover. Since both charts are so similar and essentially tell the same story, except for daytime clear, the amount of cloud cover was not integrated into the data. Instead, it was reflected by applying the data to the two charts for each direction and day part. Pasquill's graphs also reflect the decreased dispersion of toxins when there is greater than 80% daytime cloud cover. The exposure numbers in our analysis for daytime greater than 50% cloud cover, however, do not reflect the increased exposure from greater than 80% cloud cover. Therefore the actual daytime exposure numbers are higher. While the analysis in this disclosure included >80% cloud cover within >50% cloud cover, the methodology could specifically reflect >80% and other percents of cloud cover. Additionally, the methodology could combine the two nighttime charts into a single chart and the two daytime charts into a single chart or present the data in a different way.

For each of the four directions and day-part (night, day), the analysis calculated the number of hours that the data from two or more consecutive hours reached the threshold for the weather conditions identified in the chart and the number of days and the number of nights there was at least one alert-hour downwind of the emitting facility. Two consecutive hours with weather conditions that reach the threshold is identified as one alert-hour; three consecutive hours with weather conditions that reach the threshold is identified as two alert-hours; four consecutive hours with weather conditions that reach the threshold is identified as three alert-hours, etc. This method can also be applied to a single hour or more being the threshold.

The charts in this disclosure show the amount of toxins from a source annually emitting 40,000 lbs and 20,000 lbs of VOCs at various distances from the source and at various wind speeds and cloud cover. The method can be applied to any amount of VOCs or other airborne chemical or mix of chemicals and can present data from more than one nearby emitting source.

To adapt Pasquill's airborne toxin concentration analyses to VOC dispersion, a base case was calculated showing the expected concentration of VOCs during the day and during the night at less than 50% and at greater than 50% cloud cover at four wind speeds for seven locations ranging from 100 meters to 10 km downwind from a pollution source.

The Pasquill graphs configured for this disclosure assume flat terrain. Hills and valleys can modify the airflow resulting in a shift regarding exposure location with, for example, increased exposure at 3 km and less at 1 km; or, increased exposure to those living south-southeast and less to those living south when the wind is from the north. The methodology can be modified to integrate terrain characteristics.

### Sensitivity Classes and Frequency of Short-Term Serious Health Risk

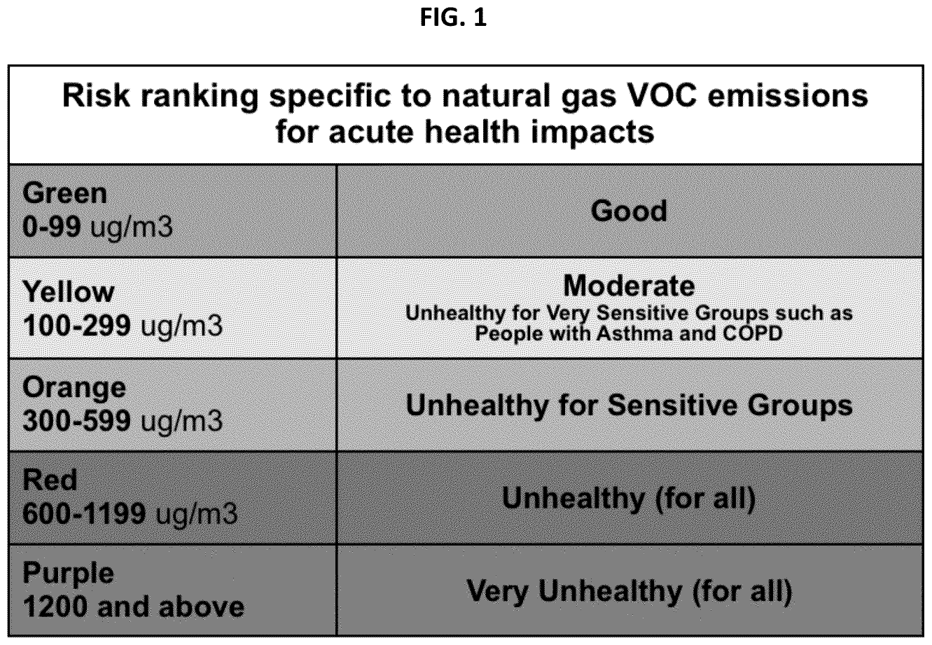

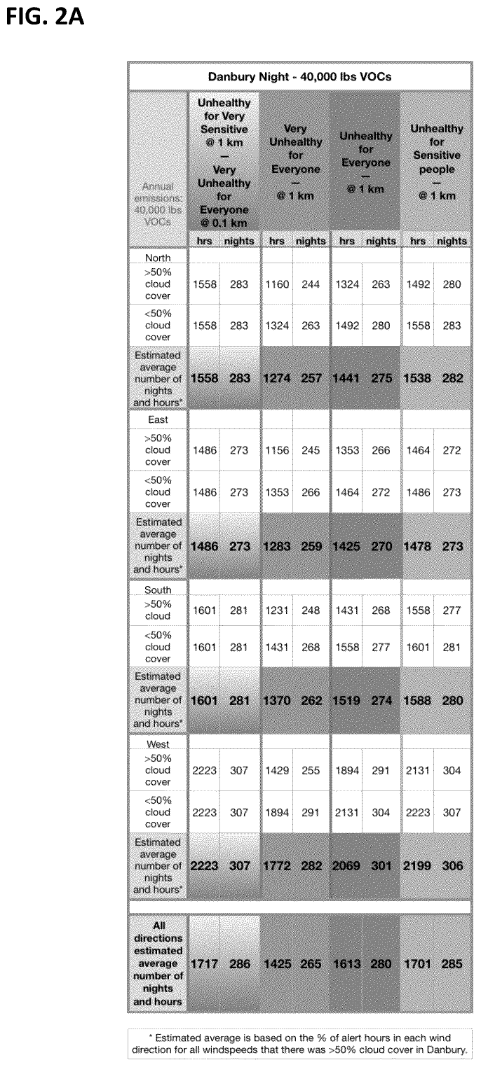

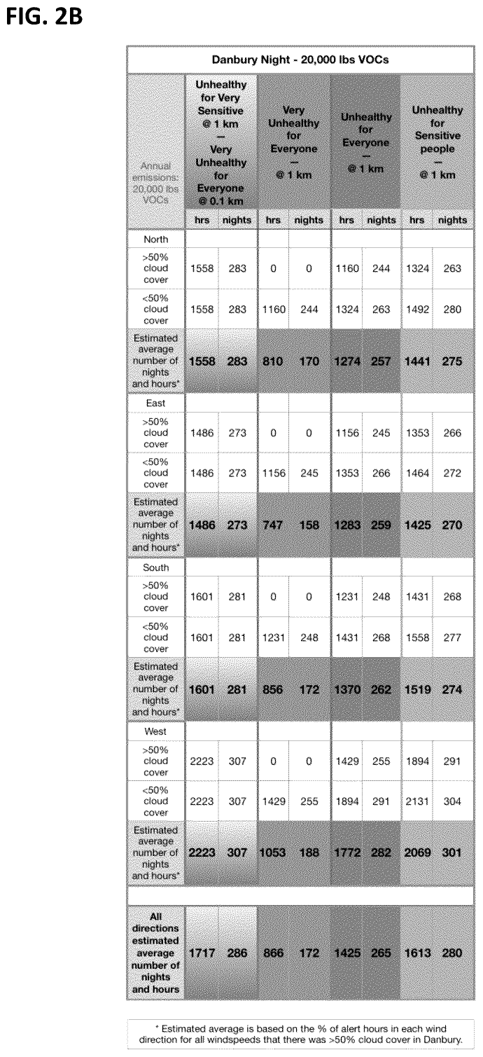

FIG. 1 shows, by color-coding, levels of an individual's sensitivity to natural gas VOC emissions. FIGS. 2A-2B and FIGS. 3A-3E show the frequency of exposure to an unhealthy concentration of VOCs. The numbers in the colored boxes indicate the number of alert-hours and the number of days or nights that someone of that color's sensitivity or where noted on the charts someone more sensitive, living at that distance from the facility could be breathing air that exceeds the health recommendations for their sensitivity group. The implementation shown in FIG. 1 can be modified to reflect impact from other airborne chemicals and chronic health impacts.

The numbers in the Impact by Sensitivity Group charts shown in FIGS. 2A-2B show the number of hours, days and nights that a person of a particular sensitivity group should be aware of and cautioned about the expected concentration of toxins. For each wind direction, the numbers on one line indicate greater than 50% cloud cover and the numbers on the other line indicate less than 50% cloud cover (which includes clear). The colored box for each direction is a weighted average of the frequency in that wind direction that the cloud cover was greater than or less than 50%. The bottom line of the chart is the average of the numbers in the colored wind direction boxes.

### Estimation Profiles of Residential Exposures and Weather Factors

Hourly residential exposure estimates in this disclosure are based on our reference air model chart and the 40,000 lbs of VOC emissions reported to New York State. The level, timing and frequency of the local exposures were determined over a one-year period based on hourly NOAA weather data for the location. The weather data on percent cloud cover, wind speed and wind direction each hour day or night was used to determine stability class and direction of plume impacts. The 40,000 lbs of emissions was compared to the reference emissions to adjust the levels of local residential exposures for each of the 112 categories (boxes) on each wind direction's Exposure Frequency Risk chart. These were based on the source and the air stability category. Air stability is a function of wind speed and cloud cover.

The number of hours and days that the emissions would impact the residents were calculated at each of five hazard levels (green, yellow, orange, red, purple) and shown in the risk table. The hazard level calculated for VOCs was patterned after the EPA hazard categories and are described below.

### Process for Ranking Exposure Risks for the Mixtures of VOCS in Peak Air Exposures

This disclosure focuses on the non-cancer health effects elicited by short inhalation exposures to mixtures of VOCs, with exposures of a minimum of 2 consecutive hours. The health effects elicited by the mixture are a function of the proportion of each chemical in the mixture and its potency. While this disclosure used as its basis the mix of VOCs emitted at fracked gas extraction, storage and transport sites, the methodology can be applied to any airborne chemical toxins from any type of emitting source in any type of industry. Four categories of VOCs were found in the emissions: straight and branched chains that do not contain substitutions of active groups, substituted alkanes such as acids, aldehydes, glycols and ketones, halogenated compounds, and aromatic hydrocarbons such as benzene, toluene and PAHs. The short chain, c1 to c10 carbon chemicals predominate in the mixtures.

A scale of human toxic potency for each of the chemicals in such mixtures has been developed for workers and current standards for both long-term exposures and immediate acute impact from short-term exposures are published by NIOSH in the Guide to Chemical Hazards [57]. This disclosure used as its basis NIOSH's short-term exposure guidelines. Qualitative and other quantitative lists of the chemicals in the emissions were reviewed. NIOSH's target organ and potency recommendations for each of the four categories of chemicals in the mixture were examined. Chemicals with similar target organs and potencies were grouped together. This method can be applied to chronic exposures. The example shown in FIG. 1 can be modified to reflect chronic exposures.

These groups included acute risks to the eyes, ears, nose and throat, the respiratory system, cardiovascular system and central nervous system. The potencies for each of the compounds in the NIOSH handbook range from air concentrations of 1200 ug/m3 down to 10 ug/m3 with toxic potencies clustered between 100 and 300 μg/m3.

Based on this finding, a risk ranking was constructed for the different short-term exposures to the mixture. Chloromethane, the most toxic commonly identified chemical was used as a guide and surrogate. Its threshold guidance for workplace exposures is 100 ppm or 202 ug/m3. The risk was adjusted for children, the elderly and other susceptible persons by dividing this number by 2. The scaling system used is similar to that used by EPA for criteria pollutants. Reference to actions of higher exposures to chloromethane was used to guide ranking for the higher exposures to the mixtures. For emissions of other chemical mixes, this method can use one or more different chemicals as a guide and surrogate.

FIG. 1 shows the risk ranking using EPA's format and descriptions for air quality exposure categories of individual chemicals. Thus a table was constructed to evaluate the immediate and short-term health risks from peak exposures to the specific mixture of compounds. The risk follows the federal workplace and indoor air guidance standards.

The Exposure Frequency Risk charts are based on the 5 chemicals that according to the NYS DEC make up 95% of the VOC emissions. There are other chemicals in the emissions that this disclosure does not account for but could.

Comparison of Weather Model Estimates with Actual Emissions

To determine the accuracy of the weather model results, this disclosure compared the day and hour of peak VOC exposures for the four months March through June 2022 from a monitor, ½ km southwest of the compressor station used in this disclosure, with day and hour exposures identified in the disclosure's weather model. Not only did an analysis of peak exposures indicate that night had significantly more peaks than day, the date and hourly times matched 87% of the time. A different brand continuous air monitor can be used.

### Results

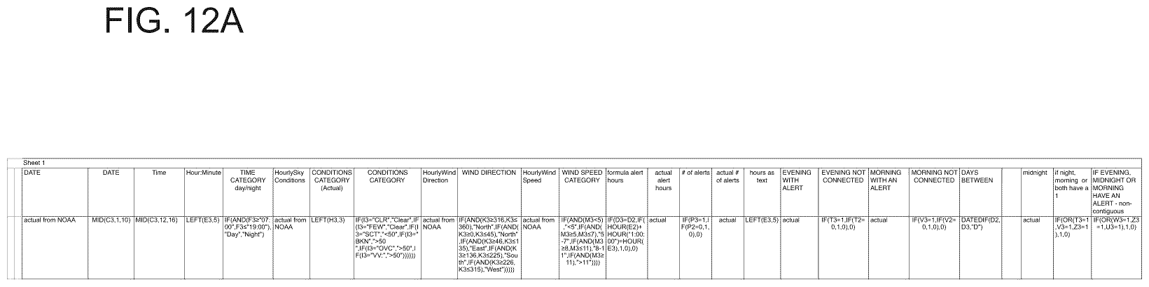

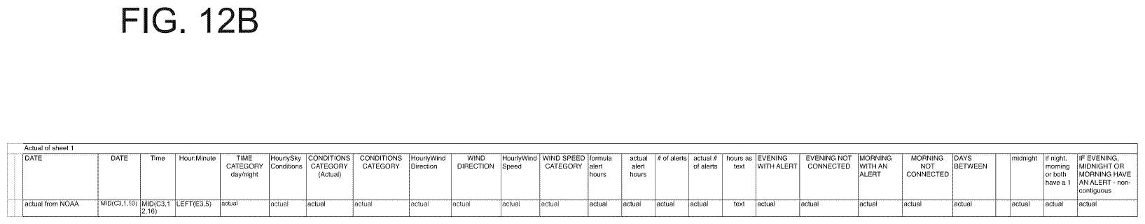

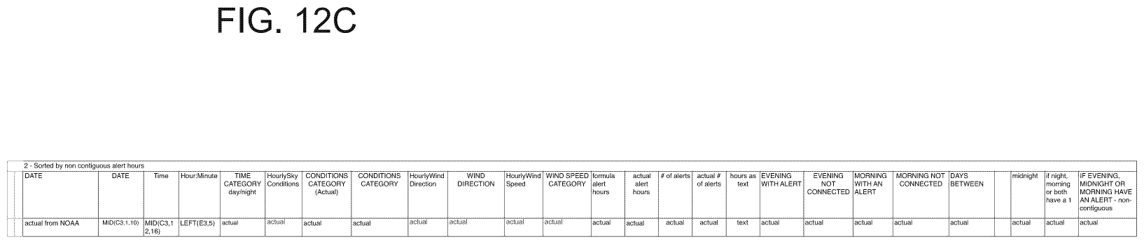

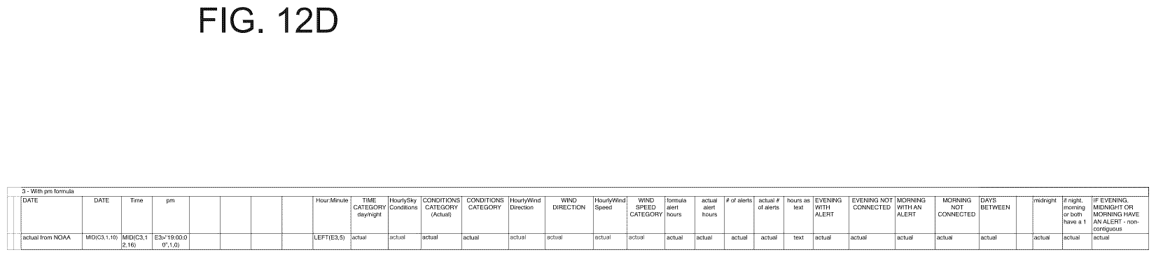

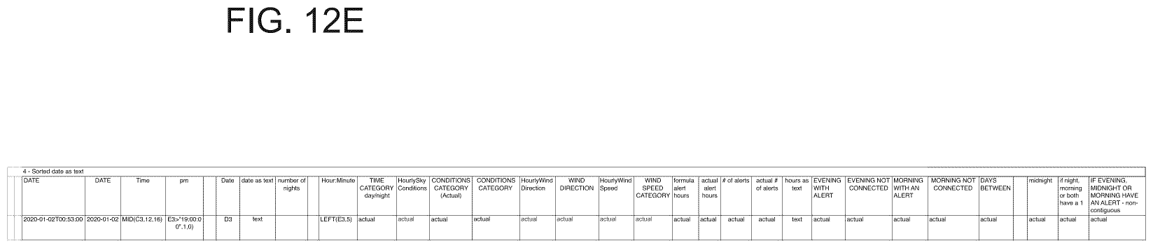

This disclosure's Impact by Sensitivity Group charts (FIGS. 2A-2B) and its VOC Exposure Frequency Risk charts (FIGS. 3A-3E) show risk frequencies for individuals with varying sensitivities. One set of formulas for performing the calculations for Exposure Frequency Risk as described herein are shown in FIG. 12A-12E. Other formulas for performing the calculations give the same results.

The short-term health impacts for exposure to natural gas VOCs are described in FIG. 1, the risk guideline chart. The following charts show frequency of short-term health hazardous exposures from a facility in New York annually emitting 40,000 lbs of VOCs. Although VOCs can cause cancer, birth defects, neurological damage and chronic health impacts those conditions are not considered in these short-term acute health effect charts. The data from this methodology, however, can be used when considering chronic health impacts.

FIGS. 2A-2B show tables displaying the number of hours and nights that people in different sensitivity groups (very sensitive, sensitive, everyone) living different directions and distances from a gas facility annually emitting 40,000 lbs of VOCs and 20,000 lbs of VOCs would be exposed to VOCs in concentrations considered unhealthy for them.

The yellow/purple column shows the number of hours and nights that the emissions would be Unhealthy for a Very Sensitive person living 1 km from the emitting facility and the number of hours and nights it would be Very Unhealthy for Everyone living 0.1 km from the emitting facility. The purple column shows the number of hours and nights it would be Very Unhealthy for Everyone living 1 km from the emitting facility. The red column shows the number of hours and nights it would be Unhealthy for Everyone living 1 km from the emitting facility and the orange column shows the number of hours and nights it would be Unhealthy for Sensitive Groups 1 km away. A different presentation of the results can be used.

### Exposure Frequency Risk Charts

Of the Exposure Frequency Risk charts, FIGS. 3A-3D show the effect of wind speed, cloud cover, day part (day/night) and dilution distance on frequency and intensity of exposures for emissions of 40,000 lbs of VOCs to people with different health conditions living at specific distances from the emitting facility. Each wind direction of North, East, South, and West is respectively shown on FIGS. 3A-3D. The colored boxes reflect the impact to each sensitivity level.

The charts show the frequency of unhealthy exposures (purple to yellow) and the frequency of high exposures day and night at distances from 0.1 km to 10 km from the source. The charts indicate that at night the high exposures extend further from the source than during the day and that the hazard is higher at low wind speeds and during calms. The charts also show that individuals who are more sensitive are affected at distances farther from the source than those who are less sensitive. The charts present the number of alert hours a year in each direction. An alert hour is 2 consecutive hours that the weather data meet the threshold for an unhealthy impact so there is a minimum of 60 minutes meeting the exposure risk threshold.

As illustrated in the Exposure Frequency Risk charts described herein, those who live closer to an emitting site typically will more frequently be exposed to unhealthy air. This is the result of the greater concentration of pollutants in the air near the emitting facility because they have not yet been dispersed by the wind and/or the sun's heat.

In most cases as the wind blows stronger, the density of the emissions in the air become more diluted. However, when the daytime wind blows stronger, the reverse occurs for nearby locations because more pollutants are carried from the emitting facility.

During the day, cloud cover increases toxic concentration while at night there is higher toxic concentration when there is less cloud cover.

The Exposure Frequency charts described herein show a number of main observations:
influence of wind direction
influence of wind speed
influence of cloud cover
influence of day and night
influence of stagnant air (the dark vertical line indicates that for no wind direction and no windspeed, toxins are considered to be present in all four directions within 1 km)

The findings in the charts show:
For people living close to these emitting facilities, the projected toxic concentration of the air at night from facility emissions frequently exceeds acceptable VOC risk.
Most nights both sensitive and non-sensitive people who are 1 km (0.6 miles) or less from a facility with 40,000 lbs/year of VOC emissions are exposed to an unhealthy amount of toxins regardless of wind speed or cloud cover. During the day there is greater dilution so unhealthy exposures are reduced in frequency and distance.
At night when the wind is not strong, many of those who are farther from the emitting site frequently are exposed to an unhealthy amount of toxins.
At night toxic concentration of air 1 km or less from facilities annually emitting only 20,000 lbs of VOCs exceeds acceptable VOC risk guidance for these chemicals for both sensitive and non-sensitive people the vast majority of nights.

The analysis found that at night both sensitive and non-sensitive people who are 1 km from the sample facility used in this disclosure or a comparable facility at this location are exposed to an unhealthy amount of toxins over 270 nights/year (5 out of 7 nights) and for over 1400 to 2000 nighttime hours depending on direction relative to the emitting facility.

Due to greater vertical dilution during the day, relative to night, there are far fewer days and hours exceeding acceptable risk limits.

If the compressor station were to reduce annual VOC emissions to 20,000 lbs, residents 1 km from the site would still be exposed to an unhealthy amount of toxins over about 250 to 280 nights and over 1270 to 1770 nighttime hours depending on direction relative to the emitting facility. This reduction would reduce frequency of risk that the nighttime air is very unhealthy for everyone at 1 km by about 35% but has only a minor risk reduction for people who are more sensitive.

When the disclosure's toxic concentration model, using as the source term 40,000 lbs of gas compressor station VOCs, was applied to a comparison site in the southwest, Albuquerque, the findings were site specific but exposures were also frequent.

### Multiple Hour Exposures

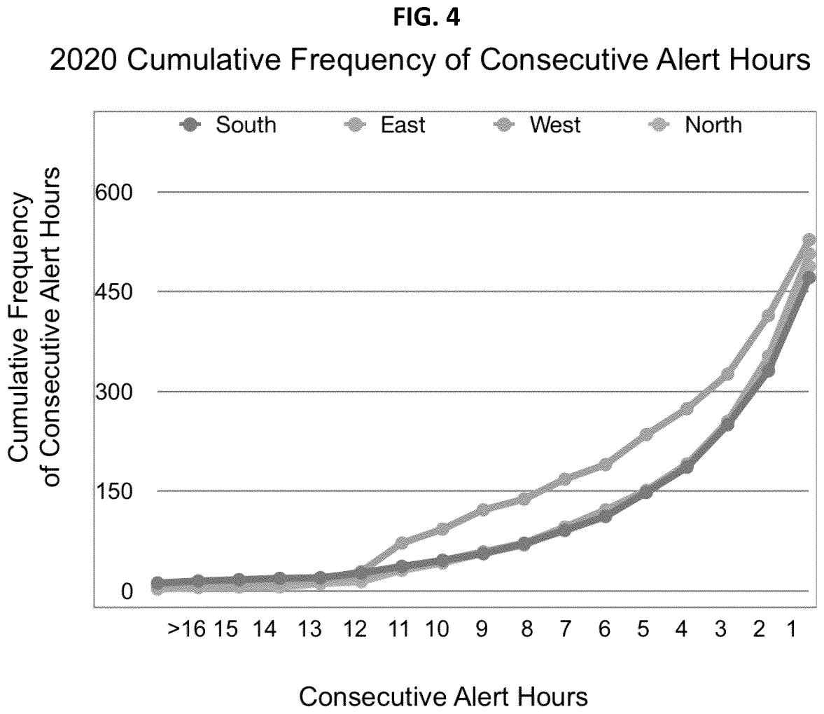

As shown in FIG. 4, in addition to brief exposures, there are frequent exposures lasting multiple hours. For example, when the wind was coming from the west, there were 138 times that there were 8 or more consecutive alert hours of toxic concentrations that posed a hazardous health risk. When the wind was coming from the east, south and north, there were about 70 times for each direction that there were 8 or more consecutive alert hours.

The greater than 16 consecutive alert hour data point includes 15 periods with 17-19 consecutive alert hours; 9 periods with 20-29 consecutive alert hours; 3 periods with 30-31 consecutive alert hours; and, 1 period of 45 consecutive alert hours.

Comparison of Weather-Based Hourly Predictions and the Monitored Data for Wind from the Northeast

The high nighttime frequency of hazardous pollution is confirmed by the hour-specific frequency of peak concentrations from three VOC monitors positioned southwest of the sample facility used in this disclosure.

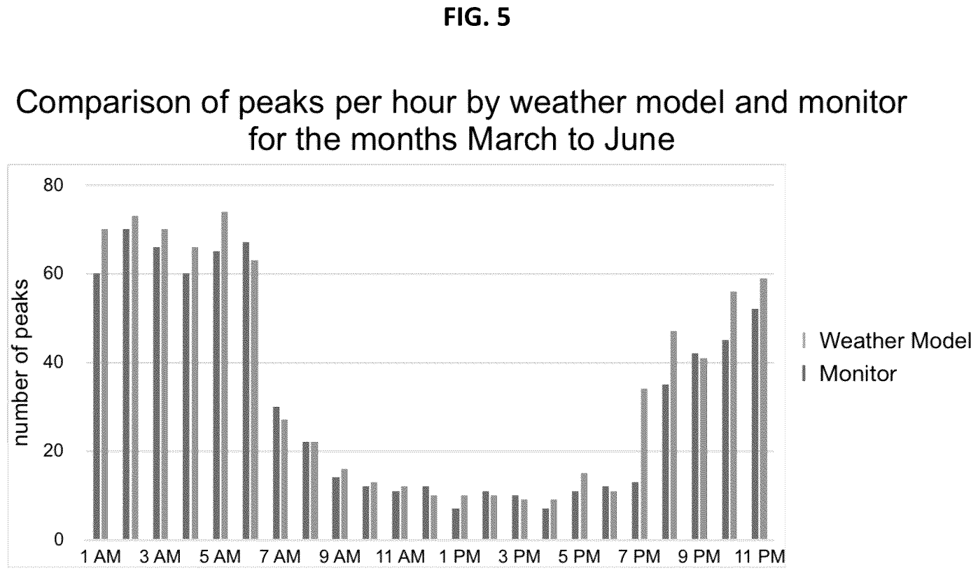

The hourly observed peak emissions from the closest monitor ½ km from the compressor station was compared with the weather model's hours meeting the exposure threshold for March to June. Data from this monitor continuously recording VOCs was used to identify hourly periods that were three times over baseline level. FIG. 5 shows the observed peak frequency from the monitor (blue) and frequency of high exposure hours based on the weather model (orange). These findings also show that the chance of a high exposure is greater at night than during the day.

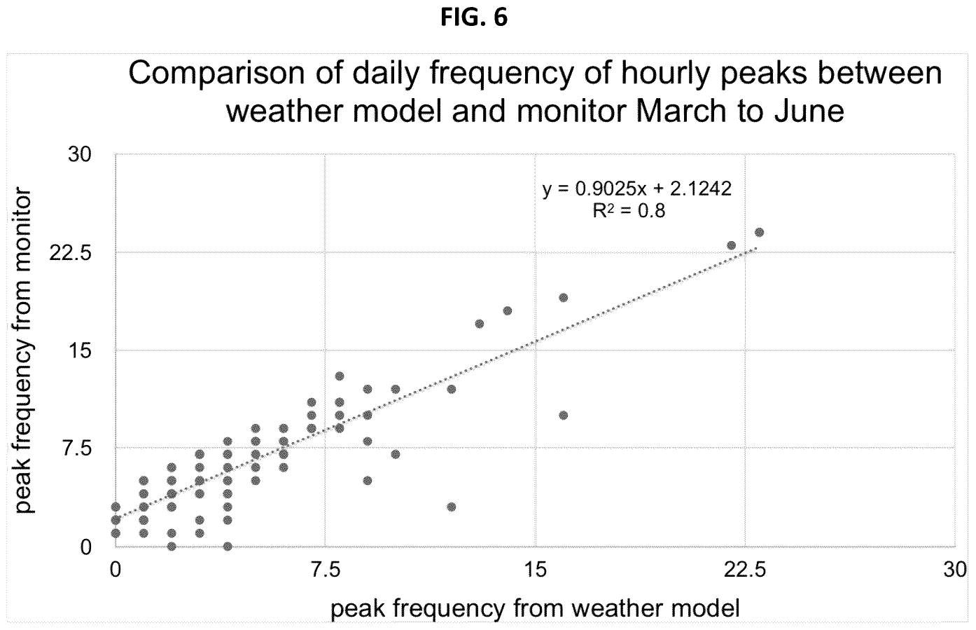

FIG. 6 compares the number of daily hourly peaks by weather model and monitor for the months of March through June. Its R² value is 0.8. An hour by hour analysis of the weather model's alert-hours and no alert hours indicated that they were the same hours as the monitor's peak and no peak hours 87% of time.

### Discussion

The findings from this disclosure show that residences up to and possibly over 5 km of a gas compressor station emitting 40,000 lbs of VOCs experience multiple periods of high exposures to a mixture of VOCs both outside and inside their homes over one year. While this disclosure used the VOC emissions from a natural gas compressor station in New York State as the basis for its analysis, this methodology can be applied to other pollutants or polluting facilities.

The periods of high exposure of the emitted chemicals are due to 5 conditions, wind direction, wind speed, cloud cover, distance from the sources and time of day or night. Substantial cloud cover and low wind speeds reduce the dilution and increase the concentrations of pollutants at locations close to the emitting source.

For this disclosure, the New York State Department of Environmental Conservation provided the data on the components and amounts in the emissions mixture. NOAA provided the weather data. Risk-ranking scales needed to determine the acute health hazards from the exposure to the VOCs in mixtures was based on the NIOSH Guide to Chemical Hazards and peer reviewed reports.

The compressor station near Danbury Connecticut reported emitting 40,000 pounds of VOCs in 2020. Classes of VOCs in the pollutants emitted included light weight alkanes, aldehydes, halogenated hydrocarbons, aromatics and PAHs. Overall there were 18 different VOCs reported in the mixtures. These VOCs are associated with 5 major acute health conditions, asthma, COPD, sensory and cognitive health effects and cardiovascular attacks. In addition, these VOCs can cause chronic effects including cancer, birth defects, pulmonary and endocrine system conditions. The risk ranking used was based on relative concentrations, potencies and actions of the 5 predominate VOCs. PMs, CO and NOx were also present in the mixtures emitted but were not included in the risk evaluation. The weather would impact the concentration of those toxins and others in the same way it impacts VOCs. Consequently, the toxicity of the air for nearby residents is greater than that shown in the Exposure Frequency Risk charts described herein.

The Exposure Frequency Risk charts show that everyone at 1 km or less from the compressor station could have experienced air that was unhealthy for them an average of 1425-2069 nighttime hours and 270-301 nights depending on direction relative to the emitting source. Sensitive people at 1 km or less would have had a risk of a health response 1478-2199 nighttime hours in 2020. (FIGS. 3A-3E).

Although less frequent, the model results show that periods of high exposures can occur up to 6 miles from the compressor station, usually these exposures occur at night due to low air dilution (FIGS. 3A-3E).

In 2020 there were 3042 residents within one mile of the compressor station and 283 within one half mile who were frequently exposed to high levels of toxins. Nationally there are over 17 million people who live near natural gas facilities as well as many millions more who live near other polluting facilities who also experience high exposures.

The methodology can apply to more than a facility's actual emissions. It can also be used to quantify the exposure frequency and toxic concentrations in the air in scenarios where the facility was emitting more or emitting less toxins. For example, if the compressor station used in this disclosure were to reduce emissions to only 20,000 lbs of VOCs annually, residents 1 km from the site would still be exposed to an unhealthy amount of toxins over about 250 to 280 nights and for over 1270 to 1770 nighttime hours depending on direction relative to the emitting facility. During the day when there is greater vertical dilution, widespread unacceptable risks from 20,000 lbs of VOCs apply more to those living ½ km from the facility. Relative to night, there are far fewer days and hours exceeding acceptable risk limits. Reducing emissions by 50% only reduces frequency of risk by about 35%.

Acute health risks to residents near natural gas compressor stations and other toxin-emitting facilities have been reported. The current outdoor air quality health risk assessments under-estimate the acute risks. The EPA's regional NAAQS are not designed to evaluate exposures to residents in close proximity to emitting facilities and thereby fail to recognize the need to protect them and the need to reduce the amounts of VOCs and other toxins emitted.

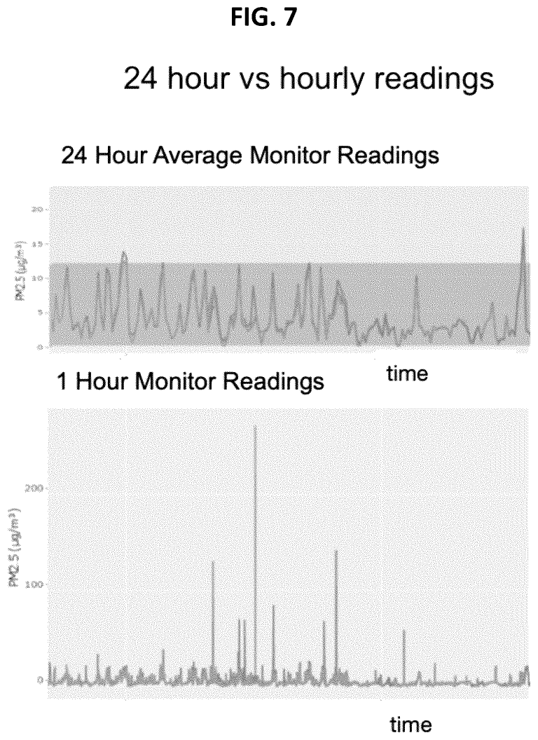

The EPA's NAAQS are the basis for the federal government and most states' emissions regulations [8-10]. NAAQS average the air pollution regionally and over time so do not reflect the toxicity of the air for people living in close proximity. As shown in FIG. 7, averaging masks the toxic peaks.

The EPA has established an environmental risk ranking for VOCs, but, although necessary, it has not established an overall health risk ranking for VOCs. While different mixtures in the emissions have different component chemicals, it is possible to systematically evaluate the risks of mixtures if the relative quantity and hazard of each VOC component were considered.

The risk ranking approach used in this disclosure revealed some interesting points. For example, the estimated exposure from the concentration of the over 4,000 lbs of formaldehyde emitted annually at the sample facility used in this disclosure exceeds the NIOSH work place standard for formaldehyde. (Data not shown). Secondly, present in the mixtures were other synergists such as PM 2.5 and PM fines which would increase the potency assumptions but not the specific actions of the chemicals present. Their impact was not included in the quantification used in this disclosure, but could be. The methodology of this disclosure includes the ability to measure the cumulative impact of multiple chemicals.

High exposures within houses will occur due to intrusion of the VOC emissions from hourly exchange of outside and inside air, at a typical rate of about ½ to 2 air changes per hour, which after 3 hours will approach outside concentrations [58]. There are many times when the high concentration exposures last more than three hours (FIG. 4). In those cases, the toxic concentration inside the home becomes an issue. At a rate of one air change of outside and inside air per hour, after one hour, the concentration inside the home is one half the concentration outside the home. After two hours, the inside concentration rises to 75% of that of the outside air. After three hours the inside concentration is nearly 88% or nearly the same as the outside. As shown in FIG. 4, there are multiple periods of sequential hours with high VOC concentrations thereby causing the inside air concentration to equal outside concentration and therefore being a health hazard.

Modeled High Exposures Compared with Monitor Peak Concentrations

An air monitor ½ km southwest of the sample facility used in this disclosure continuously measured VOCs from March through June 2022. The monitor measures a reference VOC. While a direct quantitative comparison would require the monitor to measure the exact chemicals in the emitted mixture, the air dilutions should be the same and show when same peaks occur. Consequently, it was possible to compare the times when peaks occurred and the number of peaks per day. A comparison of hours when modeled high exposures occurred show similar hourly patterns as the monitor's findings (FIG. 5). The peaks per day (FIG. 6) illustrate the high percent of times that the high exposures predicted by the model and the peaks observed on the monitor occurred at the exact same hour on the same day. When the rate of emissions is relatively constant, this would be expected since high toxic concentrations are driven by weather components. A comparison of the specific hours when the weather model indicated a high exposure and the monitor indicated a peak and when neither indicated one, had a correlation of 87%.

### CONCLUSIONS

1. Archived local weather information can be used to characterize and show the frequency of hazardous exposure risks both past and future near point sources such as compressor stations.

2. This disclosure shows that the frequency of high concentrations of VOCs vary with time of day and weather conditions. Everyone at 1 km or less from the mid-sized NY compressor station used in this disclosure would have been exposed to air that was unhealthy for them an average of 1425-2069 nighttime hours and 270-301 nights depending on direction of their home relative to the emitting source. People sensitive to lower concentrations who live at 1 km or less would have had a risk of a health response 1478-2199 nighttime hours in 2020. (FIGS. 2A-2B).

3. Ongoing and short-term exposure to airborne toxins can cause many health impacts including asthma, COPD and other pulmonary and cardiovascular diseases.

4. Currently the predominantly used methodology for assessing exposures and guiding regulations is the EPA's NAAQS. The NAAQS' methodology, however, averages toxins regionally rather than hyper-locally and over multiple hours instead of hourly so it does not identify the frequent high exposures to residents near emitting sources and therefore does not adequately protect the tens of millions of Americans who live in close proximity to these sources.

5. Polluting facilities tend to be in lower income communities. Peak exposures and frequent high concentrations of toxins that residents in these communities breathe are more damaging than the lower averaged concentrations assessed by the NAAQS would indicate. The health cost and human suffering caused by this regulatory oversight is high and represents a serious environmental justice situation.

In at least one embodiment, a display interface can render a display of the information produced by the components of the system 100, including the charts and graphs shown in FIGS. 1-7. In at least one embodiment, dashboard display can be presented on a client computer accessed over network. In at least one embodiment, user interfaces can be employed without departing from the spirit and/or scope of the claimed subject matter. Such user interfaces can have any number of user interface elements, which can be arranged in various ways. In some embodiments, user interfaces can be generated using web pages, mobile applications, GIS visualization tools, mapping interfaces, emails, file servers, PDF documents, text messages, or the like. In at least one embodiment, the system can include processes and/or API's for generating user interfaces.

For example, as shown in the flow chart 200 of FIG. 11, feed data can be combined into an aggregated data set and visualized using an interface 202, for example a GIS visualization tool (e.g.: Mapbox, CARTO, ArcGIS, or Google Maps API) or other interfaces. In an embodiment, the system configured to provide exposure risk insights and interfaces 202 therefor is described with respect to exemplary data processing of weather and environmental event data as described herein. An interface can also be configured to output data via interfaces to downstream devices such as client computers. As shown in FIG. 8, the data feeds can include exemplary feeds such as, for example data set 204, data set 206, and event data 206.

Embodiments described with respect to systems are described in conjunction with FIGS. 1-11, can be implemented by and/or executed on a single network computer. In other embodiments, these processes or portions of these processes can be implemented by and/or executed on a plurality of network computers. Likewise, in at least one embodiment, processes described with respect to systems or portions thereof, can be operative on one or more various combinations of network computers, client computers, virtual machines, or the like can be utilized. Further, in at least one embodiment, the processes described in conjunction with FIGS. 1-11 can be operative in system with logical architectures such as those also described herein.

It will be understood that each block of the flowchart illustration, and combinations of blocks in the flowchart illustration, can be implemented by computer program instructions. These program instructions can be provided to a processor to produce a machine, such that the instructions, which execute on the processor, create means for implementing the actions specified in the flowchart block or blocks. The computer program instructions can be executed by a processor to cause a series of operational steps to be performed by the processor to produce a computer-implemented process such that the instructions, which execute on the processor to provide steps for implementing the actions specified in the flowchart block or blocks. The computer program instructions can also cause at least some of the operational steps shown in the blocks of the flowchart to be performed in parallel. Moreover, some of the steps can also be performed across more than one processor, such as might arise in a multi-processor computer system or even a group of multiple computer systems. In addition, one or more blocks or combinations of blocks in the flowchart illustration can also be performed concurrently with other blocks or combinations of blocks, or even in a different sequence than illustrated without departing from the scope or spirit of the disclosure.

Accordingly, blocks of the flowchart illustration support combinations for performing the specified actions, combinations of steps for performing the specified actions and program instruction means for performing the specified actions. It will also be understood that each block of the flowchart illustration, and combinations of blocks in the flowchart illustration, can be implemented by special purpose hardware-based systems, which perform the specified actions or steps, or combinations of special purpose hardware and computer instructions. The foregoing example should not be construed as limiting and/or exhaustive, but rather, an illustrative use case to show an implementation of at least one of the various embodiments.

### REFERENCES

1. Oil and Gas Threat Map. Is your health threatened? 17.3 million live within ½ mile of oil and gas production {internet} 2022 <https://oilandgasthreatmap.com/>.
2. Czolowski E D, Santoro R L, Srebotnjak T, Shonkoff S B C. Toward consistent methodology to quantify populations in proximity to oil and gas development: A national spatial analysis and review. Environ Health Perspect. 2017; 125(8):086004.
3. USEPA. USA Environmental Protection Agency-US NAAQS National Ambient Air Quality Services Accessed Dec. 18, 2022 {internet} 2021 https://www.epa.gov/criteria-air-pollutants/naaqs-table.
4. Bachmann J. Will the circle be unbroken: a history of the U.S. National Ambient Air Quality Standards. Journal of the Air & Waste Management Association. 2007; 57(6):652-97.
5. Owens E O, Patel M M, Kirrane E, Long T C, Brown J, Cote I, et al. Framework for assessing causality of air pollution-related health effects for reviews of the National Ambient Air Quality Standards. Regul Toxicol Pharmacol. 2017; 88:332-7.
6. Thakrar S B, S., Adams P. Azevedo, I. Muller, N, Pandkis, S. Polasky, S. Pope, A. Robinson, A. Aprte, J. Tessum, C. Marshall, J. Hill, J. Reducing mortality from air pollution in the United States by targeting specific emission sources. Environ Science Techn Lett. 2020; 7:639-45.
7. Enerdata Yearbook 2021 Natural Gas Production https://yearbook.enerdata.net/natural-gas/world-natural-gas-production-statistics.html. 2021.
8. Energy Information Agency. Natural Gas Explained here our natural gas comes from https://www.eia.gov/energyexplained/natural-gas/where-our-natural-gas-comes-from.php. 2021.
9. Powell T. Is your natural gas actually fracked Sightline Institute Available at https://www.sightline.org/2017/10/30/is-your-natural-gas-actually-fracked/. 2017.
10. Tschofen P, Azevedo I L, Muller N Z. Fine particulate matter damages and value added in the US economy. Proc Natl Acad Sci U.S.A. 2019; 116(40):19857-62.
11. Curtis L. PM(2.5), NO(2), wildfires, and other environmental exposures are linked to higher Covid 19 incidence, severity, and death rates. Environmental Science and Pollution Research International. 2021; 28(39):54429-47.
12. Zang S T, Luan J, Li L, Yu H X, Wu Q J, Chang Q et al. Ambient air pollution and COVID-19 risk: Evidence from 35 observational studies. Environ Res. 2021; 204:112065.
13. Lembo R, Landoni G, Cianfanelli L, Frontera A. Air pollutants and SARS-CoV-2 in 33 European countries. Acta Bio-Medica: Atenei Parmensis. 2021; 92(1):e2021166.
14. Liu N, Bu Z, Liu W, Kan H, Zhao Z, Deng F, et al. Health effects of exposure to indoor volatile organic compounds from 1980 to 2017: A systematic review and meta-analysis. Indoor Air. 2022; 32(5):e13038.
15. Russo P N, Carpenter D O. Air Emissions from natural gas facilities in New York State. International Journal of Environmental Research and Public Health. 2019; 16(9).
16. Wickliffe J S, T. Howard, J. Frahm, E. Increased long-term health risks attributable to select volatile organic compounds in residential indoor air in southeast Louisiana. 2020; Nature (10):62149.
17. Ware J H, Spengler J D, Neas L M, Samet J M, Wagner G R, Coultas D, et al. Respiratory and irritant health effects of ambient volatile organic compounds. The Kanawha County Health Study. American Journal of Epidemiology. 1993; 137(12):1287-301.
18. Brown D R, Lewis C, Weinberger B I. Human exposure to unconventional natural gas development: A public health demonstration of periodic high exposure to chemical mixtures in ambient air. J Environ Sci Health A Tox Hazard Subst Environ Eng. 2015; 50(5):460-72.
19. Martin KAVL, E. Hilbert, T. Pollit, K. Survey of airborne organic compounds in residential communities near a natural gas compressor station: Response to community concern. Environmental Advances. 2021; 5.
20. Zielinska B, Campbell D, Samburova V. Impact of emissions from natural gas production facilities on ambient air quality in the Barnett Shale area: a pilot study. Journal of the Air & Waste Management Association (1995). 2014; 64(12):1369-83.
21. Dockery D W, Pope C A, 3rd, Xu X, Spengler J D, Ware J H, Fay M E, et al. An association between air pollution and mortality in six U.S. cities. N Engl J Med. 1993; 329(24):1753-9.
22. Krewski D, Burnett R, Jerrett M, Pope C A, Rainham D, Calle E, et al. Mortality and long-term exposure to ambient air pollution: ongoing analyses based on the American Cancer Society cohort. J Toxicol Environ Health A. 2005; 68(13-14):1093-109.
23. Alford K L, Kumar N. Pulmonary health effects of indoor volatile organic compounds—A Meta-Analysis. International Journal of Environmental Research and Public Health. 2021; 18(4).
24. Rumchev K, Brown H, Spickett J. Volatile organic compounds: do they present a risk to our health? Reviews on Environmental Health. 2007; 22(1):39-55.
25. Arif A A, Shah S M. Association between personal exposure to volatile organic compounds and asthma among US adult population. Int Arch Occup Environ Health. 2007; 80(8):711-9.
26. Russo A C, D. Russo & Carpenter|Institute for Health & the Environment|Rensselaer NY|{internet} 2017. Health effects associated with stack chemical emissions from NYS natural gas compressor stations: 2008-2014A Technical report prepared for the Southwest Pennsylvania Environmental Health Project underwritten by the Park Foundation 12 Oct. 2017. Available at, https://www.albany.edu/web/about/assets/Complete report.pdf
27. Allen D T. Atmospheric emissions and air quality impacts from natural gas production and use. Annual Review of Chemical and Biomolecular Engineering. 2014; 5:55-75.
28. Fann N, Baker K R, Chan E A W, Eyth A, Macpherson A, Miller E, et al. Assessing human health PM (2.5) and ozone impacts from U.S. oil and natural gas sector emissions in 2025. Environmental Science & Technology. 2018; 52(15):8095-103.
29. Field R A, Soltis J, Murphy S. Air quality concerns of unconventional oil and natural gas production. Environmental Science Processes & Impacts. 2014; 16(5):954-69.
30. Moore C W, Zielinska B, Pétron G, Jackson R B. Air impacts of increased natural gas acquisition, processing, and use: a critical review. Environmental Science & Technology. 2014; 48(15):8349-59.
31. Bamber A M, Hasanali S H, Nair A S, Watkins S M, Vigil D I, Van Dyke M, et al. A systematic review of the epidemiologic literature assessing health outcomes in populations living near oil and natural gas operations: Study quality and future recommendations. International Journal of Environmental Research and Public Health. 2019; 16(12).
32. Carpenter D O. Hydraulic fracturing for natural gas: impact on health and environment. Reviews on Environmental Health. 2016; 31(1):47-51.
33. Wollin K M, Damm G, Foth H, Freyberger A, Gebel T, Mangerich A, et al. Critical evaluation of human health risks due to hydraulic fracturing in natural gas and petroleum production. Arch Toxicol. 2020; 94(4):967-1016.
34. Mckenzie L M, Witter R Z, Newman L S, Adgate J L. Human health risk assessment of air emissions from development of unconventional natural gas resources. The Science of the Total Environment. 2012; 424:79-87.
35. Paulik L B, Donald C E, Smith B W, Tidwell L G, Hobbie K A, Kincl L, et al. Emissions of polycyclic aromatic hydrocarbons from natural gas extraction into air. Environmental Science & Technology. 2016; 50(14):7921-9.
36. Peng R D, Liu J C, McCormack M C, Mickley L J, Bell M L. Estimating the health effects of environmental mixtures using principal stratification. Statistics in Medicine. 2022.
37. Vardoulakis S, Giagloglou E, Steinle S, Davis A, Sleeuwenhoek A, Galea K S, et al. Indoor exposure to selected air pollutants in the home environment: A systematic review. International Journal of Environmental Research and Public Health. 2020; 17(23).
38. Helmig D. Air quality impacts from oil and natural gas development in Colorado. Elm Sci Anth. 2020; 8(4):1-22.
39. Brown D R, Greiner L H, Weinberger B I, Walleigh L, Glaser D. Assessing exposure to unconventional natural gas development: using an air pollution dispersal screening model to predict new-onset respiratory symptoms. J Environ Sci Health A Tox Hazard Subst Environ Eng. 2019; 54(14):1357-63.
40. Bushong A, Mckeon T, Regina Boland M, Field J. Publicly available data reveals association between asthma hospitalizations and unconventional natural gas development in Pennsylvania. PLOS One. 2022; 17(3):e0265513.
41. Blinn H N, Utz R M, Greiner L H, Brown D R. Exposure assessment of adults living near unconventional oil and natural gas development and reported health symptoms in southwest Pennsylvania, USA. PLOS One. 2020; 15(8):e0237325.
42. Casey J A, Savitz D A, Rasmussen S G, Ogburn E L, Pollak J, Mercer D G, et al. Unconventional natural gas development and birth outcomes in Pennsylvania, USA. Epidemiology. 2016; 27(2):163-72.
43. Mckenzie L M, Guo R, Witter R Z, Savitz D A, Newman L S, Adgate J L. Birth outcomes and maternal residential proximity to natural gas development in rural Colorado. Environ Health Perspect. 2014; 122(4):412-7.
44. Stacy S L, Brink L L, Larkin J C, Sadovsky Y, Goldstein B D, Pitt B R, et al. Perinatal outcomes and unconventional natural gas operations in Southwest Pennsylvania. PLOS One. 2015; 10(6):e0126425.
45. Whitworth K W, Marshall A K, Symanski E. Maternal residential proximity to unconventional gas development and perinatal outcomes among a diverse urban population in Texas. PLOS One. 2017; 12(7): e0180966.
46. Mckenzie L M, Allshouse W B, Byers T E, Bedrick E J, Serdar B, Adgate J L. Childhood hematologic cancer and residential proximity to oil and gas development. PLOS One. 2017; 12(2):e0170423.
47. Tustin A W, Hirsch A G, Rasmussen S G, Casey J A, Bandeen-Roche K, Schwartz B S. Associations between unconventional natural gas development and nasal and sinus, migraine headache, and fatigue symptoms in Pennsylvania. Environ Health Perspect. 2016.
48. Graham J, Irving J, Tang X, Sellers S, Crisp J, Horwitz D, et al. Increased traffic accident rates associated with shale gas drilling in Pennsylvania. Acid Anal Prev. 2015; 74:203-9.
49. Casey J A, Wilcox H C, Hirsch A G, Pollak J, Schwartz B S. Associations of unconventional natural gas development with depression symptoms and disordered sleep in Pennsylvania. Sci Rep. 2018; 8(1):11375.
50. Jemielita T, Gerton G L, Neidell M, Chillrud S, Yan B, Stute M, et al. Unconventional gas and oil drilling is associated with increased hospital utilization rates. PLOS One. 2015; 10(7):e0131093.
51. McMullin T S, Bamber A M, Bon D, Vigil D I, Van Dyke M. Exposures and health risks from volatile organic compounds in communities located near oil and gas exploration and production activities in Colorado (U.S.A.). International Journal of Environmental Research and Public Health. 2018; 15(7).
52. Kahl D C, H. Atmospheric stability characterization using the Pasquill Method: A critical evaluation. Atmos Environment. 2018; 187:196-209.
53. Pasquill, F. Atmospheric diffusion: The dispersion of windborne material from industrial and other sources.: D. Van Norstand Company, Ltd: London, 1962.
54. Wikipedia. Atmospheric Dispersion Modeling, From Wikipedia—The Free Encyclopedia {internet} 2022. https://en.wikipedia.org/wiki/Atmospheric_dispersion_modeling.
55. NOAA. Climate Data Online: National Oceanic and Atmospheric Administration {internet} 2022. https://www.ncei.noaa.gov/cdo-web/.
56. New York State. NYS DEC Contaminant Totals List 2020.-3373000060/AES2020 ALGONQUIN GAS SOUTHEAST COMPRESSOR STATION. Air.Inventory@dec.ny.gov.
57. Department of Health and Human Services, Centers for Disease Control and Prevention, National Institute for Occupational Safety and Health, NIOSH Pocket Guide to Chemical Hazards, https://www.cdc.gov/niosh/docs/2005-149/pdfs/2005-149.pdf. 2007.
58. EPA. US EPA Exposure Factors Handbook, https: www.epa.gov/exponox/about-exposure-factors-handbook. 2011.

1. A system comprising a memory including program instructions and a processor configured to execute instructions for a method comprising:
inputting hourly or other period of weather data for a period of time;
mapping and overlaying the hourly or other period of weather data onto air dispersion charts based on air dispersion graphs that calculate toxin concentration at discreet distances;
identifying a frequency for discreet distances where a toxin concentration exceeds an acceptable risk threshold for people of varying health conditions and attendant sensitivities to toxins.

2. The system of claim 1, wherein the memory includes program instructions and the processor configured to execute the instructions for the method further comprising:
calculating annual emissions of a plurality of airborne chemicals.

3. The system of claim 2, wherein the memory includes program instructions and the processor configured to execute the instructions for the method further comprising:
identifying hours from the hourly weather data that meet or exceed the risk threshold; and
comparing the hours or other period from the hourly or other period of weather data that show peak levels of airborne chemicals as determined by a continuous air monitor.
